(19) United States
(12) Patent Application Publication
(10) Pub. No.: US 2025/0044219 A1
(43) Pub. Date: Feb. 6, 2025

(54) SINGLE-PIXEL MULTISPECTRAL IMAGER FOR FLARE AND BURNER COMBUSTION EFFICIENCY MEASUREMENT

(71) Applicant: Schlumberger Technology Corporation, Sugar Land, TX (US)

(72) Inventor: Sebastien Catheline, Clamart (FR)

(21) Appl. No.: 18/791,502

(22) Filed: Aug. 1, 2024

Related U.S. Application Data
(60) Provisional application No. 63/517,349, filed on Aug. 3, 2023.

(51) Int. Cl. G01N 21/25; G01N 21/31; G01N 33/00

(52) U.S. Cl. CPC G01N 21/255; G01N 21/31; G01N 33/004; G01N 2201/0633; G01N 2201/105

(57) ABSTRACT

Embodiments presented provide for a method for using an imager to determine combustion efficiency measurement. In embodiments, a single-pixel multispectral imager is used to provide accurate measurements for combustion efficiency for flare and burner assemblies used in industry.

### CROSS-REFERENCE TO RELATED APPLICATIONS

The present application claims priority to U.S. Provisional Patent Application 63/517,349, filed Aug. 3, 2023, the entirety of which is incorporated by reference.

### FIELD OF THE DISCLOSURE

Aspects of the disclosure relate to imaging technology. More specifically, aspects of the disclosure relate to a multispectral imager for flare and burner combustion efficiency measurement.

### BACKGROUND

Industry continually attempts to become more efficient in operations to maximize profits and minimize risks. Use of energy has been a large focus for industry for decades. By efficiently using energy, the costs to manufacture products can be drastically reduced. As time progresses, energy costs generally rise, leading to a need for innovation.

Historically, burning of hydrocarbon fuels was accomplished through a few methods. In addition to burning of hydrocarbon fuels, waste products, such as waste gas streams, may contain hydrocarbons. Emission of raw hydrocarbon fuels to the atmosphere is strictly regulated; therefore, steps are undertaken to prevent such hydrocarbons from being emitted.

One method used to treat hydrocarbon gaseous waste streams is to use a flare system to burn the hydrocarbons, rather than emit them. Such systems can be problematic. Flares may need constant “tuning” where the quantity of hydrocarbons supplied to the flare is strictly regulated. If such streams are not regulated, incomplete combustion may occur, resulting in inefficient conversion of hydrocarbon to carbon dioxide. The flare system must be closely monitored such that the feed streams are not too rich or lean, providing an efficient burning process.

Several methods may be used to quantify the combustion efficiency of a flare. In some instances, qualified spotters are used to look at the emissions and visually determine if the gaseous emissions are being properly burned. In other, more automated systems, a series of cameras may be established to provide a visual view of the combustion. Still further embodiments monitor oxygen and fuel provided to the burner system to calculate the most efficient burning possible. In such systems, automatic controls are provided to an operations control room to adjust parameters, as needed.

As time has progressed, there is a greater need to become more efficient and provide for even tighter controls of flare or burner capabilities. The conventional aspects of burning technologies do not provide a sufficient measurement of combustion efficiency.

There is a need to provide an apparatus and methods that are easy to operate and that provide superior results compared to conventional apparatus and methods.

There is a further need to provide apparatus and methods that do not have the drawbacks discussed above, namely incomplete combustion and emissions of hydrocarbons to the environment.

There is a still further need to reduce economic costs associated with operations and apparatus described above with conventional tools.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are therefore not to be considered limiting of its scope, for the disclosure may admit to other equally effective embodiments without specific recitation. Accordingly, the following summary provides just a few aspects of the description and should not be used to limit the described embodiments to a single concept.

In one example embodiment, an apparatus for analyzing a combustion source is disclosed. The apparatus may comprise:
a collimating assembly to accept light from a light source and produce a collimated beam,
a splitting assembly configured to accept the collimated beam and produce at least a first beam, which wavelength is included in a first interest band, and a second beam, which wavelength is included in a second interest band,
wherein the first beam is directed to a first detector configured to process the first beam, and
wherein the second beam is directed to a second detector configured to process the second beam.

In another example embodiment, the apparatus may comprise a first lens configured to accept light from a light source and produce a first beam. The apparatus may also comprise a second lens configured to accept the first beam and produce a second collimated beam. The apparatus may also comprise a scanning system configured to accept the second collimated beam and produce a redirected third beam. The apparatus may also comprise a broadband filter configured to accept the redirected third beam and produce a second filtered beam. The apparatus may also comprise a first beam splitter configured to accept the filtered second beam and split the filtered second beam into a first split portion and a second split portion. The apparatus may also comprise a second beam splitter configured to accept the second split portion and produce a third split portion and a fourth split portion. The apparatus may also comprise a mirror configured to direct the fourth split portion. The apparatus may also comprise at least a first narrowband filter configured to accept the first split portion and produce a first filtered split portion. The apparatus may also comprise a first portion lens configured to accept the first filtered split portion and produce a final first filtered split portion. The apparatus may also comprise a first detector configured to process the final first filtered split portion. The apparatus may also comprise a second narrowband filter configured to accept the third split portion and produce a third filtered split portion. The apparatus may also comprise a second portion lens configured to accept the third filtered split portion and produce a final third filtered split portion. The apparatus may also comprise a second detector configured to process the final third filtered split portion. The apparatus may also comprise a third narrowband filter configured to process the fourth split portion and produce a fourth filtered split portion. The apparatus may also comprise a fourth portion lens configured to process the fourth filtered split portion and produce a final fourth filtered split portion. The apparatus may also comprise a third detector configured to process the final fourth filtered split portion.

In another example embodiment, an apparatus for analyzing a combustion source, is disclosed. The apparatus may comprise a first lens configured to accept the collimated light. The apparatus may also comprise a second lens configured to accept the first beam and produce a second beam. The apparatus may also comprise a scanner configured to receive the second collimated beam and produce a third redirected beam. The apparatus may also comprise a broadband filter configured to accept the third beam and produce a fourth filtered beam. The apparatus may also comprise a diffraction grating configured to accept and process the fourth filtered beam and produce a diffracted light beam. The apparatus may also comprise a detector array configured to accept and process the diffracted collimated light beam.

In another example embodiment, a method for determining a combustion efficiency for a flare is disclosed. The method may comprise
accepting light from a light source and producing a collimated beam,
processing the collimated beam to produce at least a first beam, which wavelength is included in a first interest band, and a second beam, which wavelength is included in a second interest band,
processing the first beam and the second beam with a first detector (340) and a second detector to identify different bands of light to calculate a combustion efficiency.

In another embodiment, the method may comprise accepting a beam of light with a telescope from the flare and producing a collimated beam. The method may also comprise processing the collimated beam with a scanner to produce a redirected collimated beam. The method may also comprise accepting the redirected collimated beam at a broadband filter and producing a second filtered beam. The method may also comprise processing the second filtered beam with a diffraction grating to produce a diffracted collimated light beam. The method may also comprise processing the diffracted collimated light beam with a detector array to identify different bands of light to calculate a combustion efficiency.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are therefore not be considered limiting of its scope, for the disclosure may admit to other equally effective embodiments.

FIG. 1 is a side elevational view of a flaring system used in industry and a graph of combustion efficiency for the flaring system.

FIG. 2 is a graph of spectral bands of interest for a mid-infrared region of a combustion process being analyzed.

FIG. 3 is one example embodiment of an aspect of the disclosure with a transceiver that uses multiple detectors.

FIG. 4 is one example embodiment of an aspect of the disclosure with a transceiver using a diffraction grating and a detector array.

FIG. 5 is a side view of a prior art photovoltaic detector.

FIG. 6 is a diagram illustrating a dichroic filter principle.

FIG. 7 is a diagram of a Dichroic filter proposal in one example embodiment of the disclosure.

FIG. 8 illustrates the use of a Dichroic filter to isolate a hydrocarbon band.

FIG. 9 is an illustration of a Dichroic filter used to isolate a carbon dioxide band (around 4.26 um from the reference band (around 3.9 um).

FIG. 10 is a view of a dual wedge prism scanner.

FIG. 11 is a depiction of a fast-steering mirror scanner.

FIG. 12 is a method for performing an analysis of combustion efficiency measurement in one example embodiment of the disclosure.

To facilitate understanding, identical reference numerals have been used, where possible, to designate identical elements that are common to the FIG.s (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second” and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the FIGs. Like elements in the various FIGs will be referenced with like numbers for consistency. In the following description, numerous details are set forth to provide an understanding of various embodiments and/or features. It will be understood, however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

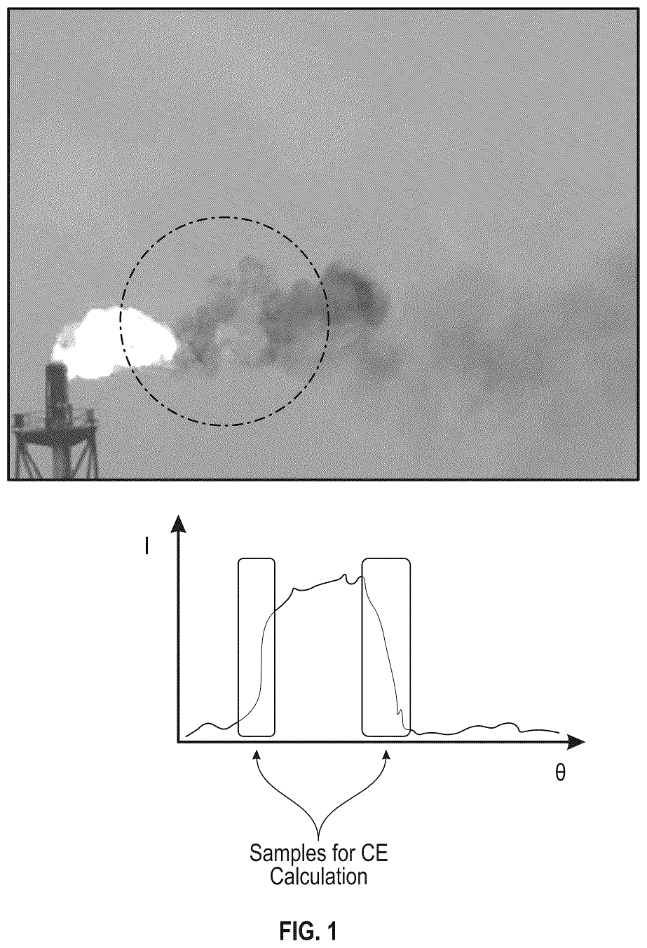

Referring to FIG. 1, aspects of the disclosure provide for a device to measure the combustion efficiency, hereinafter called “CE” of a gas flare or oil burner. At the top of FIG. 1, a flare side elevational view is provided. As illustrated, aspects of the disclosure may be where gas or oil is burned in the open atmosphere. Such non-limiting embodiments may include well test operations, oil refineries, production facilities and other petrochemical plants. Combustion efficiency is a quantity which characterizes the performance of the combustion where the goal is to convert the hydrocarbon to carbon dioxide, since it has a lower greenhouse effect than other gas hydrocarbons, such as methane. The quantity is defined as follows:

$$CE ( \% ) = 100 \frac{[ {CO}_{2} ]}{[ {CO}_{2} ] + n . [ U H C ] + [ CO ]} ,$$

but there are several other ways to define it depending on which gas species are considered. The values with the brackets refer to the concentration in parts per million (ppm), UHC stands for unburned hydrocarbon, and n. is the average number of carbon molecules in the unburned hydrocarbon. As provided at the bottom of FIG. 1, a graph showing combustion efficiency calculations is presented.

Aspects of the disclosure provide for a device that uses an imager system in the mid infrared region, between 3 and 5 um which collects light from the flame to compute the combustion efficiency (CE). The hot combustion gases in the flame within the flare or burner emit infrared light in specific wavelength bands; so, it is possible to infer the relative gas concentration in the flame from the spectral content of this light. Aspects of the disclosure operate by first collecting light from different locations in and around the flame using a scanner (steered mirror, galvo scanner—a scanner in which one or two mirrors are tilted to deflect a beam, or dual wedge prism scanner).

In the aspects described, the collected light is then split into several streams depending on wavelength. The light is then filtered to target specific spectral bands of interest such as the hydrocarbon band between 3200 nm and 3400 nm, the carbon dioxide band around 4260 nm, and a reference in between 3500 and 4000 nm. Other bands of interest can be added such as the carbon monoxide band around 4500 and 4800 nm. The individual streams of light are sent to different detectors to produce a signal proportional to the intensity in the specific spectral band. These signals are then used to compute the ratio of gas species.

The idea behind this measurement is to scan an area where a flame is present and extract the samples from the boundary of the flame where the gases are still hot so they emit light but the combustion is finished. Focusing the analysis on the boundary of the flame ensures that the CE can be calculated reliably. In embodiments, scanning may be used to generate cross section of the flame to make the detection of the flame boundary as simple as possible.

In the aspects described here, a single-pixel photon multispectral imager is used, as this imager does not produce a multi-spectral image like conventional apparatus but rather a single pixel at each sampling time. Due to the use of a scanner to aim at different targets, it is possible to reconstruct a full image of a scene in the different spectral bands of interest. Other possibilities exist than described above.

The concept of the measurement is to collect the light from in and around the flame using a telescope coupled with a scanner. When the scanner aims at the flame, the light emitted by the hot combustion gas is received and analyzed in different spectral bands. The CE can be computed in the boundary of the flame where the combustion is finished, but the combustion gases are still hot enough to emit light. By scanning across the flame, a signal intensity is observed similar to the one shown in FIG. 1. A sample on the boundary layer may be used to compute the CE.

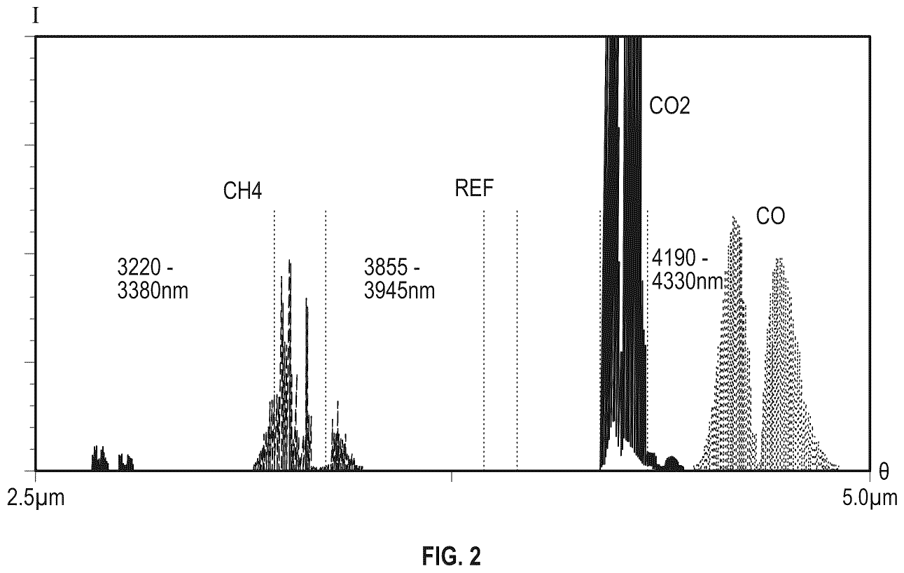

Referring to FIG. 2, the absorption coefficient of different gases of interest in the 2.5 to 5 um range is illustrated. For this measurement, we are interested mainly in the hydrocarbon band 3200-3400 nm, the carbon dioxide band 4190 to 4330 nm, a reference band somewhere between the hydrocarbon and carbon dioxide band, and if required the carbon monoxide band 4700-4800 nm. The idea of this measurement is to extract the intensity in each band of interest and compute the corresponding integrated concentration knowing the average absorption coefficient of each gas and the filters shape. Then it is possible to compute the CE. The reference band is used to correct the other intensities to remove broadband background radiation, such as the one coming from soot particles. As will be understood, other wavelength possibilities may be used than those described above.

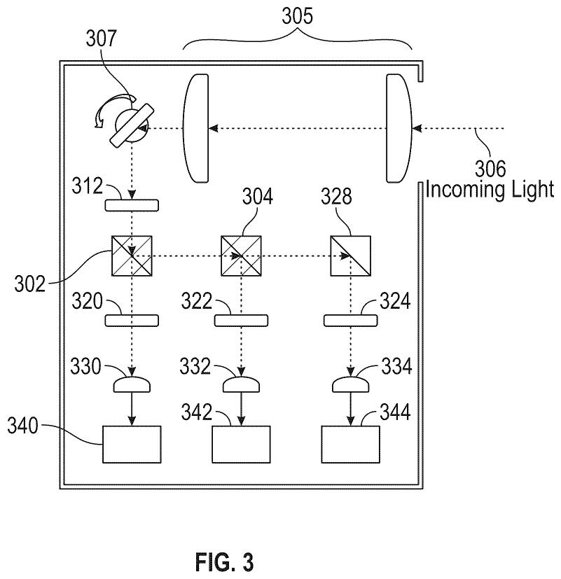

Referring to FIG. 3, an implementation of the device 300 is disclosed. Incoming light 306 is accepted by a telescope 305 and transmits the light to the scanner 307. As will be understood, various optical instruments may be used in conjunction with the device 300. As disclosed, a telescope 305 may be used. Different types of lenses within the telescope 305 may be used for help in transmitting the light. A first broadband filter 312 removes unwanted wavelengths received. In one example embodiment, a 3 to 5 um bandpass filter may be used. A specific window may be used to reject the same wavelength. The light is then separated by two beam splitters 302, 304. These beam splitters can be standard ones, meaning they are wavelength independent, or specific dichroic filters which will split the light based on the wavelength. The latter avoids wasting light which improves the signal-to-noise ratio. Each stream of light then goes through a specific “narrow” bandpass filter (100-200 nm width) targeting a specific species. The light in each stream is then focused onto a detector. Through use of the beam splitters 302, 304, and mirror 326, and associated narrowband filters 320, 322, 324, and accompanying lenses 330, 332, 334, the wavelengths impact on detectors 340, 342 and 344.

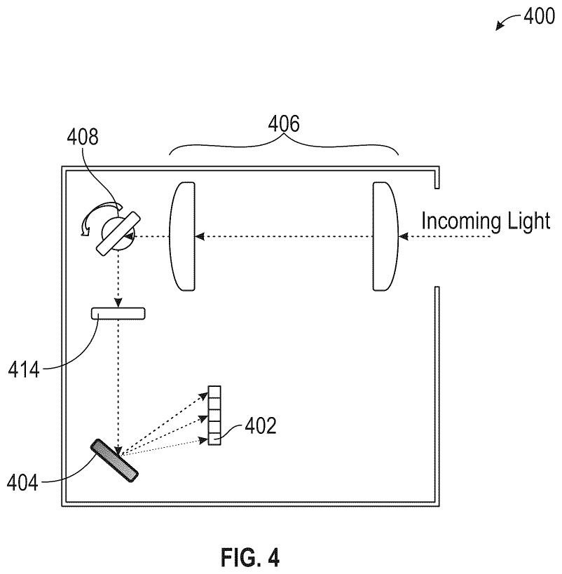

A second embodiment is illustrated in FIG. 4. In this embodiment 400, a linear detector array 402 is used instead of a single detector coupled with a diffraction grating 404. The light is diffracted by the grating based on its wavelength, which allows separation of the wavelength. The signal on each detector of the array allows computing a full spectrogram from which computations may be similarly performed for each gas. Light passes through a telescope 406 and is reflected off a scanner 408. The light then passes through a broadband filter 414, before bouncing off the diffraction grating 404 to the detector array 402. In both embodiments disclosed, a housing may be created to contain the components except the first lens. Other configurations are possible and as such, should not be considered limiting. As will be understood and as previously disclosed, various optical instruments may be used in place of the telescope 406 disclosed. In other embodiments, multiple lenses may be used within the telescope.

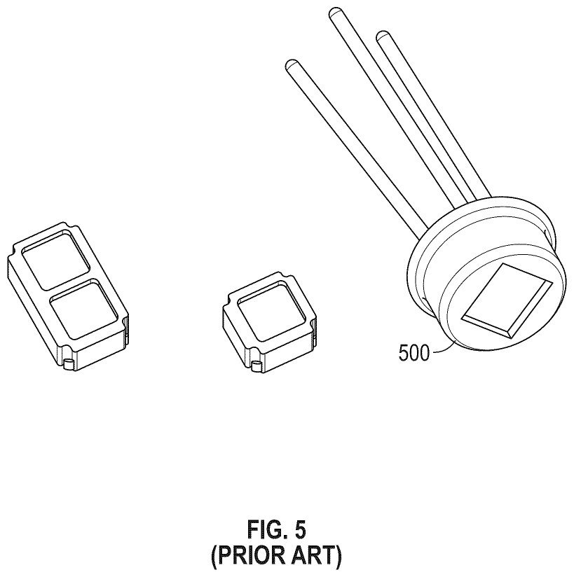

FIG. 5 shows a conventional photovoltaic detector 500 which can be used for this application because they have relatively high sensitivity, high linearity, and extremely high speed. In embodiments, a unit may be used because it includes filters for the hydrocarbon, carbon monoxide, and reference band. In addition, different types of detectors may be used, such as a non-cooled detector, which means it does not require a thermo-electric cooler. Both cooled and non-cooled detector may be used in different embodiments.

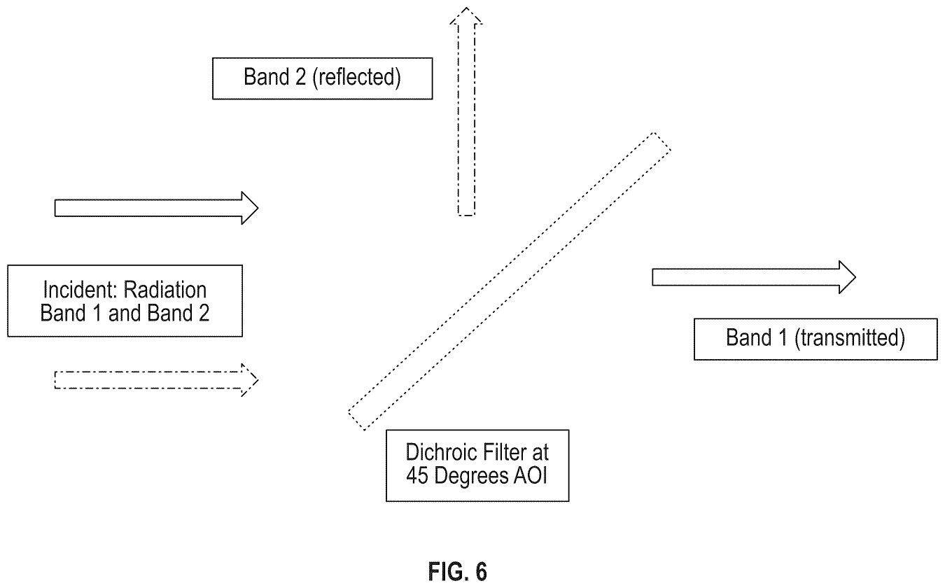

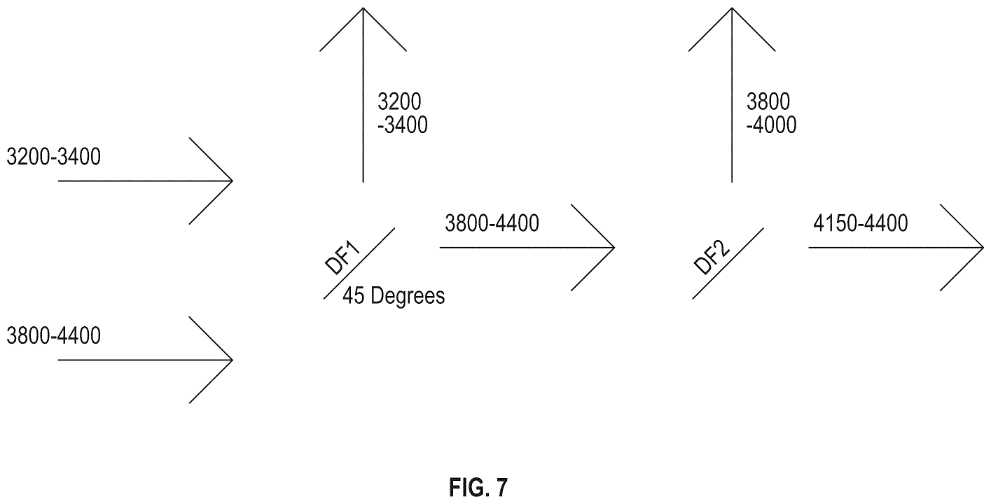

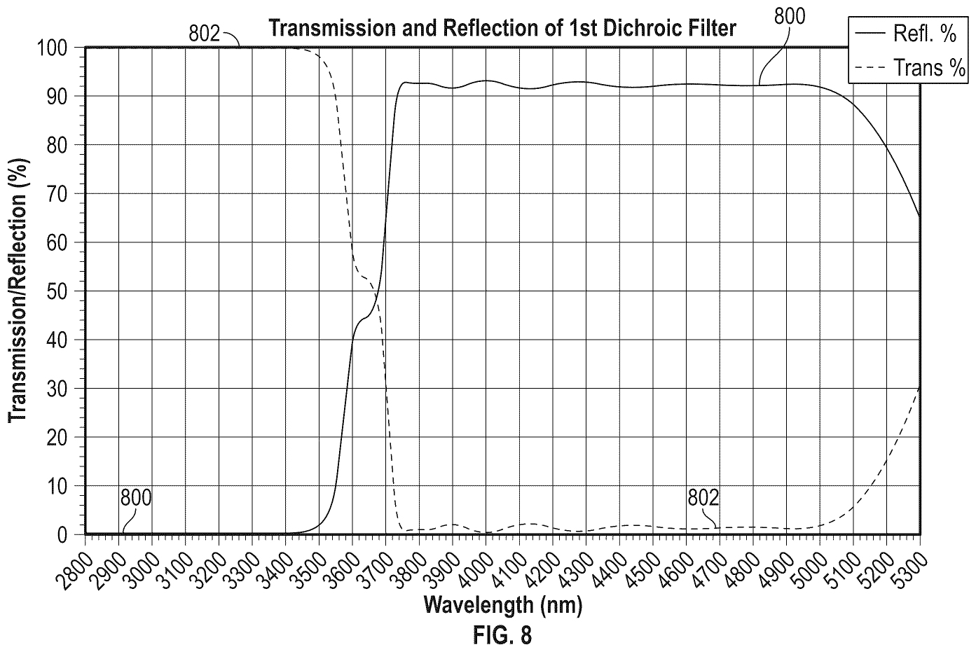

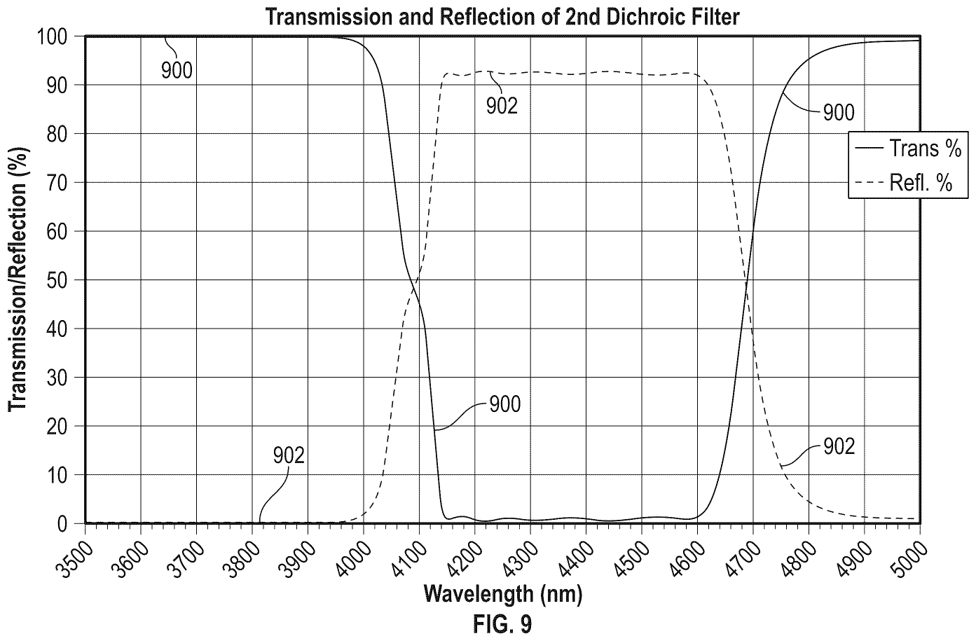

Referring to FIG. 6, arrangements using dichroic filters are disclosed. As previously discussed, the dichroic filters split the light between transmission and reflection depending on the wavelength. With these filters, all the light from the hydrocarbon and carbon dioxide bands may be extracted and sent to the detector with minimal losses. Sample detectors are shown in FIG. 7. The first filter reflects the light from the hydrocarbon band and transmits the light for the carbon dioxide and the reference bands. A second filter reflects the light from the reference band and transmits through the light from the carbon dioxide band. FIG. 8 and FIG. 9 show different types of optical coatings that may be used for these two filters. In FIG. 8, reflection percentage 800 goes from a minimum of approximately 3500 nm and then increases up to 3800 nm. Transmissibility 802 has a similar but opposite graph. In FIG. 9, reflection percentage 900 goes from a maximum of approximately 4000 nm and then decreases up to 4150 nm before increasing again at 4600 to return to a high value at 4800. Transmissibility 902 has a similar but opposite graph.

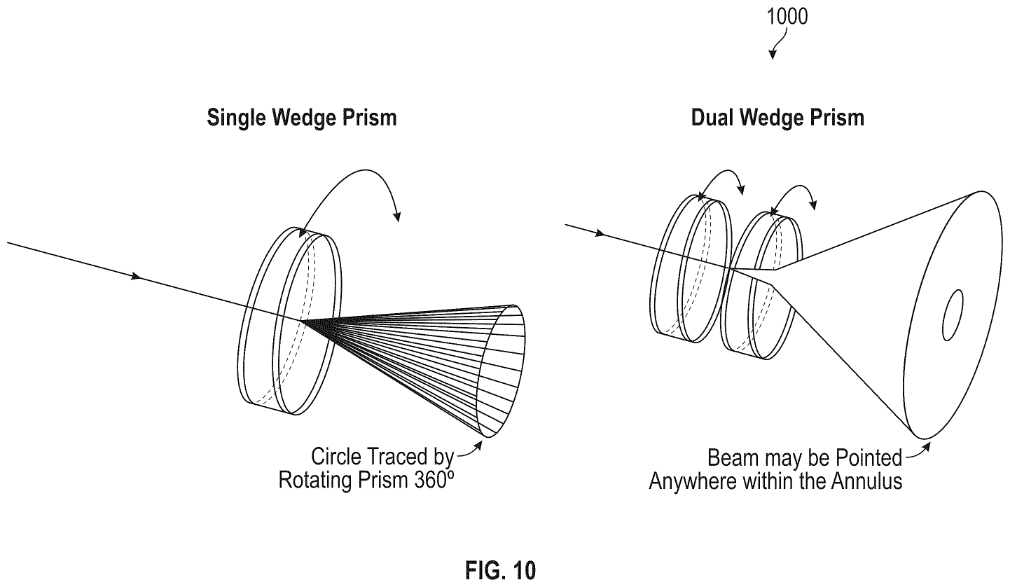

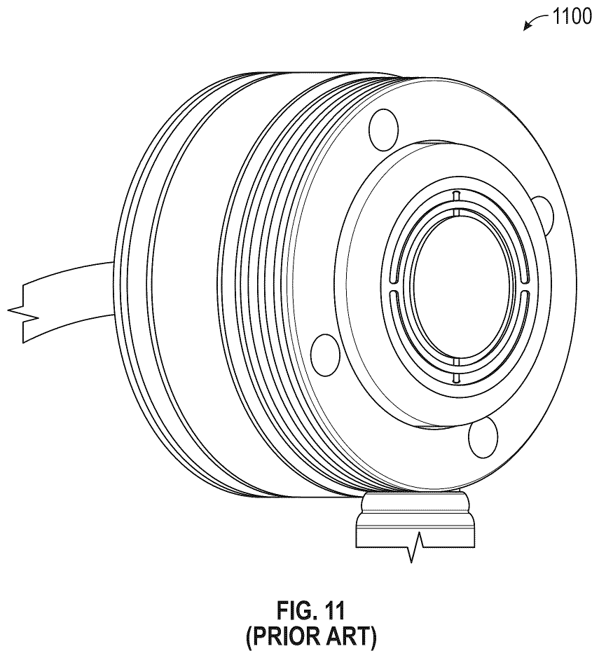

Two different arrangements may be used for the scanner. The first one is based on a dual wedge prism 1000 as shown in FIG. 10. A second solution is to use a fast-steering mirror scanner 1100 as shown in FIG. 11. In further embodiments a galvo scan or a MEMS scan may be used.

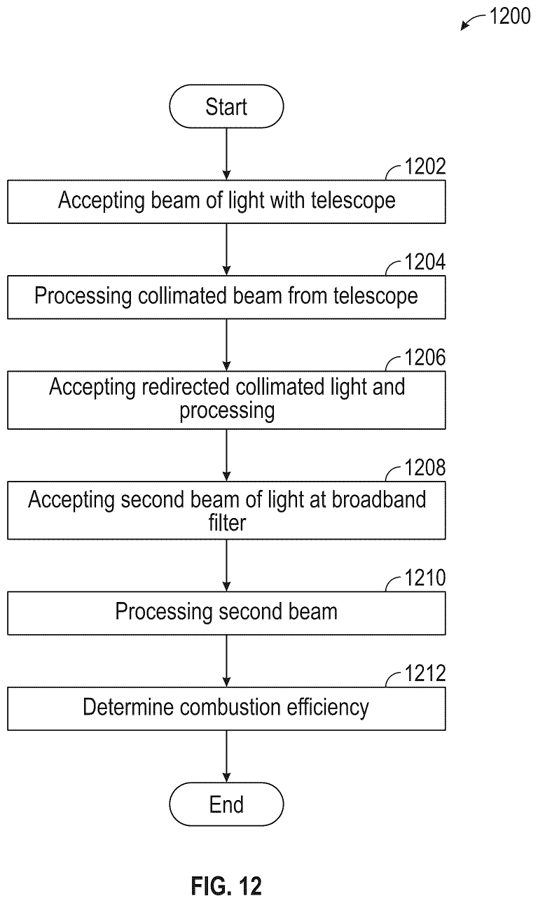

Referring to FIG. 12, a method 1200 is illustrated to determine a combustion efficiency of a combustion source. As will be understood, the combustion source may be a flare or burner as used in industry. The method 1200 may comprise, at 1202, accepting a beam of light with a telescope from the flare and producing a collimated beam. The method may further comprise, at 1204, processing the collated beam with a scanner to produce a redirected collimated light. As will be understood, the scanner may use a dual wedge prism or may be a fast-steering mirror scanner. At 1206, the method may continue with accepting the redirected collimated light at a first lens and processing the redirected collimated light to a second beam. At 1208, the method may continue with accepting the second beam at a broadband filter and producing a second filtered beam. At 1210, the method may continue with processing the second filtered beam with a diffraction grating to produce a diffracted collimated light beam. At 1212, the method may continue with processing the diffracted collimated light beam with a detector array to identify different bands of light to calculate a combustion efficiency. As will be understood, calculating the combustion efficiency may be through use of a computer that will analyze the light bands emanating from the burner or light source and then, based upon the light received, perform simulations of a flare and compare the results of the simulations and actual light bands. Data tables of such types of analysis may be referenced such that a single set of combustion efficiency calculations may be referenced to arrive at results.

Example embodiments of the claims are described herein. The example discussed should not be considered limiting of the subject matter. In one example embodiment, an apparatus for analyzing a combustion source is disclosed. The apparatus may comprise a first lens configured to accept light from a light source and produce a first beam. The apparatus may also comprise a second lens configured to accept the first beam and produce a second collimated beam. The apparatus may also comprise a scanning system configured to accept the second collimated beam and produce a redirected third beam. The apparatus may also comprise a broadband filter configured to accept the redirected third beam and produce a second filtered beam. The apparatus may also comprise a first beam splitter configured to accept the filtered second beam and split the filtered second beam into a first split portion and a second split portion. The apparatus may also comprise a second beam splitter configured to accept the second split portion and produce a third split portion and a fourth split portion. The apparatus may also comprise a mirror configured to direct the fourth split portion. The apparatus may also comprise at least a first narrowband filter configured to accept the first split portion and produce a first filtered split portion. The apparatus may also comprise a first portion lens configured to accept the first filtered split portion and produce a final first filtered split portion. The apparatus may also comprise a first detector configured to process the final first filtered split portion. The apparatus may also comprise a second narrowband filter configured to accept the third split portion and produce a third filtered split portion. The apparatus may also comprise a second portion lens configured to accept the third filtered split portion and produce a final third filtered split portion. The apparatus may also comprise a second detector configured to process the final third filtered split portion. The apparatus may also comprise a third narrowband filter configured to process the fourth split portion and produce a fourth filtered split portion. The apparatus may also comprise a fourth portion lens configured to process the fourth filtered split portion and produce a final fourth filtered split portion. The apparatus may also comprise a third detector configured to process the final fourth filtered split portion.

In another example embodiment, the apparatus may further comprise a housing configured to accept all of the components except the first lens.

In another example embodiment, the apparatus may be configured wherein the beam splitter is a dichroic mirror.

In another example embodiment, the apparatus may be configured wherein the apparatus is a transceiver.

In another example embodiment, an apparatus for analyzing a combustion source, is disclosed. The apparatus may comprise a first lens configured to accept the collimated light. The apparatus may also comprise a second lens configured to accept the first beam and produce a second beam. The apparatus may also comprise a scanner configured to receive the second collimated beam and produce a third redirected beam. The apparatus may also comprise a broadband filter configured to accept the third beam and produce a fourth filtered beam. The apparatus may also comprise a diffraction grating configured to accept and process the fourth filtered beam and produce a diffracted light beam. The apparatus may also comprise a detector array configured to accept and process the diffracted collimated light beam.

In another example, the apparatus may further comprise a housing configured to accept all of the components except the first lens.

In another example, the apparatus may be configured wherein the apparatus is a transceiver.

In another example embodiment, a method for determining a combustion efficiency for a flare is disclosed. The method may comprise accepting a beam of light with a telescope from the flare and producing a collimated beam. The method may also comprise processing the collated beam with a scanner to produce a redirected collimated light. The method may also comprise accepting the redirected collimated beam at a broadband filter and producing a second filtered beam. The method may also comprise processing the second filtered beam with a diffraction grating to produce a diffracted collimated light beam. The method may also comprise processing the diffracted collimated light beam with a detector array to identify different bands of light to calculate a combustion efficiency.

In another example embodiment, the method may be performed wherein the different bands of light are at least one of a hydrocarbon band and a carbon dioxide band.

In another example embodiment, the method may be performed wherein the scanner uses a dual wedge prism.

In another example embodiment, the method may be performed wherein the scanner uses a fast-steering mirror.

In another example embodiment, the method may be performed wherein the broadband filter is a dichroic filter.

In another example embodiment, the method may be performed wherein the scanner is a MEMS scanner.

In another example embodiment, the method may be performed wherein the scanner is a galvo scanner.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. An apparatus for analyzing a combustion source, comprising:
a collimating assembly to accept light from a light source and produce a collimated beam,
a splitting assembly configured to accept the collimated beam and produce at least a first beam, which wavelength is included in a first interest band, and a second beam, which wavelength is included in a second interest band,
wherein the first beam is directed to a first detector configured to process the first beam, and
wherein the second beam is directed to a second detector configured to process the second beam.

2. The apparatus of claim 1, wherein the splitting assembly comprises
at least a first beam splitter configured to accept the collimated beam and to split the collimated beam into a first split beam and a second split beam, and
at least a first narrowband filter and a second narrowband filter, each configured to accept a split beam and produce a filtered split beam,
wherein the first narrowband filter is configured to accept and filter the first split beam to target the first interest band, and
wherein the first beam is the filtered split beam produced by the first narrower filter and the second beam is the filtered split beam produced by the second narrower filter.

3. The apparatus of claim 2, wherein the splitting assembly further comprises
a second beam splitter configured to accept the second split beam and to split the second beam into a third split beam and a fourth split beam, and
a third narrowband filter, configured to accept a split beam and produce a filtered split beam,
wherein the second narrowband filter is configured to accept and filter the third split beam to target the second interest band,
wherein the third narrowband filter is configured to accept and filter the fourth split beam to target a third interest band,
wherein a third filtered beam produced by the third narrowband filter forms a third beam produced by the splitting assembly, which wavelength is included in the third interest band, and is directed to a third detector configured to process the third beam.

4. The apparatus of claim 2, wherein at least one beam splitter is a dichroic mirror.

5. The apparatus of claim 2, wherein the bandpass width of each narrowband filter is lower or equal to 200 nm.

6. The apparatus of claim 1,
wherein the splitting assembly comprises a diffraction grating configured to accept and process the collimated beam and produce a diffracted collimated light including the first and second beams; and
wherein the first and second detectors are parts of a detector array.

7. The apparatus of claim 1, wherein at least one interest band is a hydrocarbon band, a carbon dioxide band 4190-4330 nm, or a carbon monoxide band.

8. The apparatus of claim 1, wherein the collimating assembly comprises a telescope including a first telescope lens and a second telescope lens, the first telescope lens being configured to accept light from the light source and produce a telescope beam directed to the second telescope lens, the second telescope lens producing the collimated beam.

9. The apparatus of claim 1, comprising a scanning system configured to accept and redirect the collimated beam.

10. The apparatus of claim 9, wherein the scanning system uses a dual wedge prism or a fast-steering mirror.

11. The apparatus of claim 9, wherein the scanning system is a MEMS scanner or a galvo scanner.

12. The apparatus of claim 1, comprising a broadband filter configured to accept and filter the collimated beam.

13. The apparatus of claim 12, wherein the broadband filter is a dichroic filter.

14. A method for determining a combustion efficiency for a flare, comprising:
accepting light from a light source and producing a collimated beam,
processing the collimated beam to produce at least a first beam, which wavelength is included in a first interest band, and a second beam, which wavelength is included in a second interest band,
processing the first beam and the second beam with a first detector and a second detector to identify different bands of light to calculate a combustion efficiency.
